(19) United States
(12) Patent Application Publication
(10) Pub. No.: US 2020/0253578 A1
(43) Pub. Date: Aug. 13, 2020

(54) WEARABLE RESPIRATORY BEHAVIOR MONITORING

(71) Applicant: Arizona Board of Regents on behalf of Arizona State University, Scottsdale, AZ (US)

(72) Inventors: Junseok Chae, Scottsdale, AZ (US); Ang Chen, Tempe, AZ (US)

(21) Appl. No.: 16/783,579

(22) Filed: Feb. 6, 2020

Related U.S. Application Data
(60) Provisional application No. 62/804,351, filed on Feb. 12, 2019.

(51) Int. Cl. A61B 8/08; A61B 8/00

(52) U.S. Cl. CPC A61B 8/08; A61B 8/4472; A61B 8/4483; A61B 5/091; A61B 8/56; A61B 8/5223; A61B 8/4236; A61B 8/4477

(57) ABSTRACT

The disclosure relates to processes, articles of manufacture, devices, and systems involving biomedical monitoring measurement and analysis. More particularly, respiratory measurement, monitoring, analysis, or related systems involving wearable sensors and receiving monitors for respiratory behavior tracking and analysis.

### CROSS-REFERENCE TO RELATED APPLICATIONS

This application claims priority to U.S. provisional application 62/804,351, which was filed on Feb. 12, 2019, and is entitled Wearable Respiratory Rate Monitoring. The '351 application is incorporated, in its entirety, by reference into this application.

### TECHNICAL FIELD

Processes, articles of manufacture, devices, and systems involving biomedical monitoring, measurement and analysis are provided herein. More particularly, processes, articles of manufacture, devices, and systems involving respiratory behavior measurement, monitoring, and analysis are provided herein.

### BACKGROUND

Respiratory behavior contains relevant parameters informative of lung functionality, including: respiratory rate; profile; and volume. A commonly adopted method to characterize respiratory behavior is spirometry using a spirometer, which has been considered to be a bulky and awkward piece of equipment that requires a trained provider to understand. Spirometry is considered a physiological test measuring how an individual inhales or exhales volumes of air as a function of time. Variables measured in spirometry may be volume or flow. Spirometry is considered unsuitable for long-term continuous monitoring of respiratory behavior because of the bulk and need for constant attentive visual monitoring.

Asthma is a worldwide health problem affecting between 1-18% of the population of different countries worldwide. With asthma, hypersensitiveness of the airways causes inflammation when exposed to asthma triggers, such as common cold, stress, and changes in the weather. When triggered, the airway swells, narrowing the space for air to move in and out of lungs. If more than one typical symptom of asthma is present, the probability of having asthma increases, especially in adults. These symptoms, when described in medical terms, include forced expiratory volume delivered in the first second (FEV1) of a forced vital capacity (FVC) maneuver and the ratio of FEV1 to FVC. A reduced FEV1 may be found with many other lung diseases; however, a reduced FEV1/FVC ratio indicates airway inflammation. According to population studies, the FEV1/FVC ratio usually ranges >0.75 to 0.8 for adults and >0.9 in children. Any values less than these suggest airflow limitation. Embodiments provide advanced techniques and functions when monitoring or analyzing respiratory behavior.

### BRIEF DESCRIPTION OF THE DRAWINGS

FIG. 1 shows a wearable wireless sensor and receiving monitor as may be employed in embodiments.

FIG. 2A shows a wearable wireless sensor and distance process schematic along with a receiving monitor as may be employed in embodiments.

FIG. 2B shows additional details of a distance measurement process as may be employed in embodiments.

FIG. 2C shows a process flow schematic for a receiving monitor as may be employed in embodiments.

FIG. 3A shows a perspective view of components of a wearable wireless sensor as may be employed in embodiments.

FIG. 3B shows circuit topology of a wearable wireless sensor as may be employed in embodiments.

FIG. 3C shows operational process flows related to wireless sensor data gathering and subsequent data analysis as may be employed in embodiments.

FIG. 4A shows normalized ultrasound pressure as a function of the distance between an ultrasound emitter and an ultrasound receiver as may be employed in embodiments.

FIG. 4B shows output voltage of a wearable wireless sensor as a function of applied normalized pressure as may be employed in embodiments.

FIG. 4C shows amplitude versus time outputs of a wearable wireless sensor mounted on four locations of the chest as may be employed in embodiments.

FIG. 4D shows normalized output voltage v. time for a wearable wireless sensor when a wearer is walking and running, as may be employed in embodiments.

FIG. 5A shows a respiratory behavior plot of volume v. time, using a spirometer for reference, as may be employed in embodiments.

FIG. 5B shows a respiratory behavior plot of flow rate v. volume, using a spirometer for reference, as may be employed in embodiments.

FIG. 5C shows a respiratory behavior plot, using a wearable wireless sensor, of voltage v. time as may be employed in embodiments.

FIG. 5D shows a respiratory behavior plot, using a wearable wireless sensor, of differential voltage v. voltage as may be employed in embodiments.

FIG. 6A shows calibration testing of a wearable wireless sensor at different rotation angles, as may be employed in embodiments.

FIG. 6B shows a chart providing output data v. distance for different rotation angles of FIG. 6A, as may be employed in embodiments.

FIG. 6C shows a chart providing output data v. distance for various temperatures at the testing angles shown in FIG. 6A.

FIG. 7A shows the FEV1/FVC ratio output of a wearable wireless sensor at 2-hours, 4-hours, 6-hours, 8-hours, and 10-hours, as may be employed in embodiments.

FIG. 7B shows testing results indicative of the FEV1/FVC ratio using a spirometer and a wearable wireless sensor, as may be employed in embodiments.

FIG. 8 shows two ultrasound transducers that may be mounted on a wearable wireless sensor, as may be employed in embodiments.

### DETAILED DESCRIPTION

Embodiments may provide for processes, articles of manufacture, devices, and/or systems involving respiratory monitoring and/or analysis. Embodiments may include wearable systems using transducers movable relative to each other, or other techniques, that may monitor respiratory behavior and wirelessly send data to an external unit, such as a smartphone, or other receiving monitor for use by caregivers, other persons, or other monitoring recipients. These recipients may include personal monitoring systems as well as institutional monitoring systems. This monitoring may be used to provide real-time assistance to a wearer, to understand trends for an individual wearer, to provide long-term assistance to a wearer or cohort of wearers, to understand trends of a cohort of wearers, and for other reasons as well. Embodiments may provide quantitative information of clinically-relevant respiratory parameters such as forced vital capacity (FVC) or forced expiratory volume delivered in the first second FEV1 for better assessment of respiratory behavior of wearers.

A compact wearable system of embodiments may provide clinically-relevant parameters of respiratory behavior of wearers to caregivers, including parents, nurses, and physicians, which may allow 24/7 monitoring of those parameters outside of clinical settings, including night time. As noted above, these clinically-relevant parameters may be used for other reasons as well with regard to a wearer or for cohorts of wearers.

In embodiments, the continuous measurement of respiratory behavioral parameters may be considered to help track the progression of respiratory diseases, including asthma progression, to provide alerts to relevant caregivers to seek needed timely treatment. Embodiments may employ wireless acquisition of respiratory behavior of users. In addition to respiratory rate, embodiments may also record the respiratory profile, base the recorded tracings, and may obtain vitals parameters like FVC and FEV1/FVC ratio for a wearer or group of wearers. These parameters can be relevant to assess the progression of chronic respiratory diseases.

Embodiments may comprise a wearable wireless sensor on a paper or other substrate that may be capable of continuous monitoring of respiratory behavior and delivering clinically relevant respiratory information to a smartphone, remote monitoring system, or other receiving monitor. In embodiments, a wearable wireless sensor may be attached on the midway of the xiphoid process and the costal margin, corresponding to the abdomen-apposed rib cage of a wearer. Other attachment points are possible as well. In embodiments, a wearable sensor, may have a footprint of 40×35×6 mm³ and may weigh 6.5 g, including a 2.7 g battery. The sensor or sensors may comprise three sub-systems: i) an ultrasound emitter, ii) an ultrasound receiver and iii) a data acquisition & wireless transmitter.

A sensor, in embodiments, can be configured to convert linear strain at the wearing site to the lung volume change, by measuring the change in ultrasound pressure as a function of the distance between the emitter and the receiver. Observed or determined temporal lung volume change data, directly converted from ultrasound pressure, may be wirelessly transmitted to a smartphone, separate monitoring device, or other receiving monitor, where an application may determine and/or show an output for an observer. These outputs may comprise volume-time and flow rate-volume loop graphs, standard respiratory analysis plots or other outcomes, calculations and data of the wearer or cohorts of wearers. These calculations and data may be provided in real-time and may be stored in order to provided calculation and data for periods of time as well. The smartphone, separate monitoring device, or other receiving monitor may also be configured to analyze plots or other data and show clinically-relevant respiratory behavioral parameters, such as forced vital capacity (FVC) and forced expiratory volume delivered in the first second (FEV1). These and other determinations and parameters may be displayed on a real-time as well as on an aggregated basis.

Thus, various approaches to respiratory rate monitoring based on a wearable sensor are provided. These approaches may be based on a combination of techniques and chest/abdominal movement measurement and may promote prolonged continuous monitoring applications of wearers. Applications for the provided respiratory rate monitor system or its features may include sports, fitness, military, law enforcement, monitoring correlation of respiratory rates to hospital readmission rates, and monitoring subjects. Applications for the provided respiratory rate monitor system or its features may also include human as well as veterinary applications.

In certain embodiments, the new respiratory rate monitor may comprise two or more attachment points capable of being attached to the outside of a subject's chest or abdomen. The attachments points may be attached to the anterior or posterior of a subject's chest or abdomen. The attachment points may be attached using substances and methods suitable for attachment of medical sensors and devices, such as medical grade prosthetic adhesive and medical grade electrode adhesive. The attachment points may be attached to the chest or abdomen at an initial fixed distance apart from one another. The initial fixed distance may be the minimum distance needed for the respiratory rate monitor to register movement of the chest or abdomen. The initial fixed distance may be larger than the minimum distance needed for the respiratory rate monitor to register and/or measure movement of the chest or abdomen. The movement of the chest or abdomen registered and/or measured may be expansion, contraction, or both. The initial fixed distance may vary based on the size of the subject. The initial fixed distance may be approximately 2 to 40 inches, 3 to 10 inches, 3 to 5 inches, 3 to 15 inches, or 4 to 30 inches.

In certain embodiments, sensor attachment points may be capable of moving toward or away from each other as the chest or abdomen of a wearer moves during breathing cycles. In certain embodiments, other electrical components may also be housed in one of the attachment points. For example, the attachment point may house components that perform any or all of the functions of data collection, data storage, data processing, and display of raw or processed data. Thus, the attachment point may house any or all of one or more power source, one or more sensor, one or more light source such as an LED, one or more lens or window, one or more capacitor, one or more resistance element, one or more data processing unit such as a microprocessor, software and/or one or more algorithm, one or more data storage unit, one or more unit configured to display raw and/or processed data, and the electrical connections necessary for connecting them.

In certain embodiments, the electrical and mechanical components used or required to collect movement data and transmit it may be housed in the attachment point along with various capacitors and other circuit components, and other components, such as microprocessors, and storage, may be housed in another location. For example, any or all of one or more power source, one or more sensor, one or more capacitor, one or more resistance element, one or more transmitter or other wireless communication unit configured to transmit and/or receive data, such as a Bluetooth™ module, and the electrical connections necessary for connecting them may be housed in an attachment point. Any or all of one or more power source, one or more data processing unit such as a microprocessor, software and/or one or more algorithm, one or more data storage unit, and one or more wireless communication unit configured to receive and/or transmit data, such as a Bluetooth™ module may be housed in another location. As nonlimiting examples, any or all of one or more power source, one or more data processing unit such as a microprocessor, one or more algorithm and/or software, one or more data storage unit, and one or more wireless communication unit may be housed in a cell phone or other receiving monitor; and one or more wireless communication unit such as a Bluetooth™ module in the attachment point may be used to transmit raw data to the cell phone or other receiving monitor. A receiving monitor may therefore perform any or all of the functions of receiving data, processing it, and any or all of transmitting, storing, or displaying it. As another non-limiting example, a receiving monitor may perform only the function of receiving and transmitting data, and any or all of the functions of processing, storing, or displaying data may be performed by an appliance in the wearers' house or stable or the like, or in a doctor's office, in a clinic, in a hospital, or the like. The appliance may house any or all of one or more power source, one or more communication unit for receiving and/or transmitting data, such as a Bluetooth™ module, one or more data processing unit such as a microprocessor, software and/or one or more algorithm, and one or more data storage unit; and may perform any or all of the functions of receiving data, collection and storage of data, computation or processing of data, display of raw or processed data, or transmission of data to yet another location.

In embodiments, any wireless communication may also or alternatively be performed by a wired connection. For example, the attachment point housing the sensor may comprise a port and associated software and/or hardware for wired connection to a receiving monitor. A receiving monitor may also have a port and associated software and/or hardware for wired connection to a sensor worn by a wearer.

In embodiments, mechanical noise created during movement of the sensor may be filtered out by creating an average of a set number of samples and subtracting it from each subsequent sample. This enables the signal in certain embodiments to remain around the x-axis and better reflect the respiration pattern or other breathing cycle. In certain embodiments, to properly make sense out of the deltas calculated by the sensor, they may be added to a container variable to calculate absolute displacement. In addition, an offset reduction algorithm consisting of a running average subtracted from every sample may be implemented in the data acquisition interface.

In certain embodiments, the movement of the chest or abdomen may correlate with the respiratory rate of the wearer of the respiratory rate monitor. The correlation may be improved by removing noise from the signal. Noise may be removed from the signal using any suitable means. In embodiments, noise may be removed from the signal by averaging a set number of samples and subtracting the average from every reading.

In embodiments, the frequency and pattern observed from the displacement signal from the sensor may match the subject's breathing frequency and pattern. From the signal, the breathing frequency may be extracted by calculating the frequency of the waveform. Also, chest expansion may be obtained from the displacement values, as a longer peak would mean a greater chest expansion.

The wearable sensor may be configured using a paper substrate with electronics mounted thereon. In certain embodiments a wearable sensor may include copper-tape bridges connecting and providing power supply to an emitter. For example, BLE Nano2 may be mounted on a receiver side of a sensor to transmit collected signal to an external instrument, such as a receiving monitor via Bluetooth or another wireless communication protocol.

The wearable sensor, which may be wireless, of embodiments, may comprise piezo-material (e.g., Polyvinylidene fluoride or polyvinylidene difluoride (PVDF) film) and use this material to generate and receive ultrasound. This ultrasound may be used to monitor respiratory behaviors through measurement of the change in the circumference of the chest wall. This change in circumference has a linear relation to the volume change being tracked in embodiments. The amplitude of ultrasound may be modulated by inhale/exhale behaviors due to the changing distance between the ultrasound emitter and the receiver of a sensor or pair of sensors. With the increase and decrease of the circumference of the chest wall, causing more and less attenuation of the ultrasound pressure, the output of the wearable wireless sensor may be similarly increasing and decreasing in embodiments. This increase and decrease of ultrasound pressure may be used to determine or define the inspiration and the expiration, respectively, of a wearer. This increase and decrease of ultrasound pressure may also be used to characterize the respiratory profile by obtaining the FEV1/FVC ratio in embodiments and, therethrough, to potentially assist in asthma control of a wearer.

Sensors in embodiments may be calibrated for walking and running or other dynamic activities. Discrepancies resulting from a sensor indirectly measuring the volume change during respiration by measuring the circumference change of the chest wall may be adjusted by calibration. These discrepancies may arise from featured movements of a chest wall that may induce some indirect measurement uncertainty. Embodiments, may therefore use computational techniques to more precisely estimate volume and to convert the circumference change to volume change.

Sensor embodiments may maintain performance within reasonable range of variables due to user-induced error on sensor placement and temperature sensitivity. Suitable mean deviations of the FEV1/FVC ratio may exist in the range of 0.00%-4.25% when benchmarked by a spirometer. Other ranges may also exist and may be adjusted for through calibration. Spirometry is considered a physiological test measuring how an individual inhales or exhales volumes of air as a function of time. The primary variable measured in spirometry may be volume or flow.

A system for monitoring respiration behavior of a wearer, of embodiments, may comprise a wireless sensor, the sensor configured to output respiratory behavior data of a wearer; and a receiving monitor, the receiving monitor configured to wirelessly receive the respiratory behavior data from the wireless sensor and to determine respiratory behavior of the wearer, wherein the wireless sensor may be further configured to be surface mounted on a torso of a wearer and the wireless sensor may not completely encircle the torso of the wearer. The system may be further configured to convert linear strain data embedded in the output respiratory behavior data to lung volume change by determining the change in ultrasound pressure as a function of the distance between a first ultrasound transducer and a second ultrasound transducer of the wireless sensor. The system may also be further configured to generate an ultrasound signal by applying a non-polarized pulse stimulatory signal on a piezo-material. The system may also include piezo-material that is Polyvinylidene Difluoride (PVDF) film and the ultrasound signal may be modulated by a respiratory signal and received by a PVDF receiver of the wireless sensor. Still further, the system may also comprise a wireless sensor with a first ultrasound transducer and a second ultrasound transducer, the first transducer configured as an emitter and the second transducer configured as a receiver, the first transducer further configured to modulate a respiratory signal onto an ultrasound signal and create a modulated signal. In some instances, the first transducer may be further configured to send the modulated signal to the second transducer and in some instances the receiving monitor may be configured to determine respiratory flow rate of the wearer using the received respiratory behavior data.

A system for monitoring respiration behavior of a wearer, in embodiments, may comprising a wearable sensor, the sensor configured to output respiratory behavior data of a wearer and a receiving monitor, the receiving monitor configured to receive the respiratory behavior data from the wearable sensor and to determine respiratory behavior of the wearer, wherein the wearable sensor is further configured to be surface mounted on a torso of a wearer and the wearable sensor does not completely encircle the torso of the wearer. The system may also include the receiving monitor being configured to convert linear strain data embedded in the output respiratory behavior data to lung volume change by determining the change in ultrasound pressure as a function of the distance between a first ultrasound transducer and a second ultrasound transducer of the wearable sensor. The system may also include the wearable sensor being further configured to generate an ultrasound signal by applying a non-polarized pulse stimulatory signal on a piezo-material. the system may also include the piezo-material being Polyvinylidene Difluoride (PVDF) film and the ultrasound signal being modulated by a respiratory signal and received by a PVDF receiver of the wearable sensor. Also, the system may have the wearable sensor comprise a first ultrasound transducer and a second ultrasound transducer, the first transducer configured as an emitter and the second transducer configured as a receiver, the first transducer further configured to modulate a respiratory signal onto an ultrasound signal and create a modulated signal. Sometimes, the first transducer may be further configured to send the modulated signal to the second transducer. The system can also have the receiving monitor being configured to determine respiratory flow rate of the wearer using the received respiratory behavior data.

A system for monitoring respiration behavior of a wearer, of embodiments, may also comprise a wireless sensor, the wireless sensor configured to output respiratory behavior data of a wearer; and a receiving monitor, the receiving monitor configured to wirelessly receive the respiratory behavior data from the wireless sensor and to determine respiratory behavior of the wearer, wherein the wireless sensor is further configured to be surface mounted on a torso of a wearer and the wireless sensor comprises a plurality of ultrasound transducers, the transducers spaced a variable distance apart from each other, the distance changing during lung volume change of the wearer. The system may also include the receiving monitor being configured to convert linear strain data embedded in the output respiratory behavior data to lung volume change by determining the change in ultrasound pressure as a function of the distance between the transducers. In such systems, the wireless sensor may be further configured to generate an ultrasound signal by applying a non-polarized pulse stimulatory signal on a piezo-material. And, the system may have the piezo-material comprise Polyvinylidene Difluoride (PVDF) film and the ultrasound signal be modulated by a respiratory signal and received by a PVDF receiver of the wireless sensor. Also, the plurality of transducers may comprise a first ultrasound transducer and a second ultrasound transducer, the first transducer configured as an emitter and the second transducer configured as a receiver, the first transducer further configured to modulate a respiratory signal onto an ultrasound signal and create a modulated signal. And, in embodiments, the first transducer may be further configured to send the modulated signal to the second transducer.

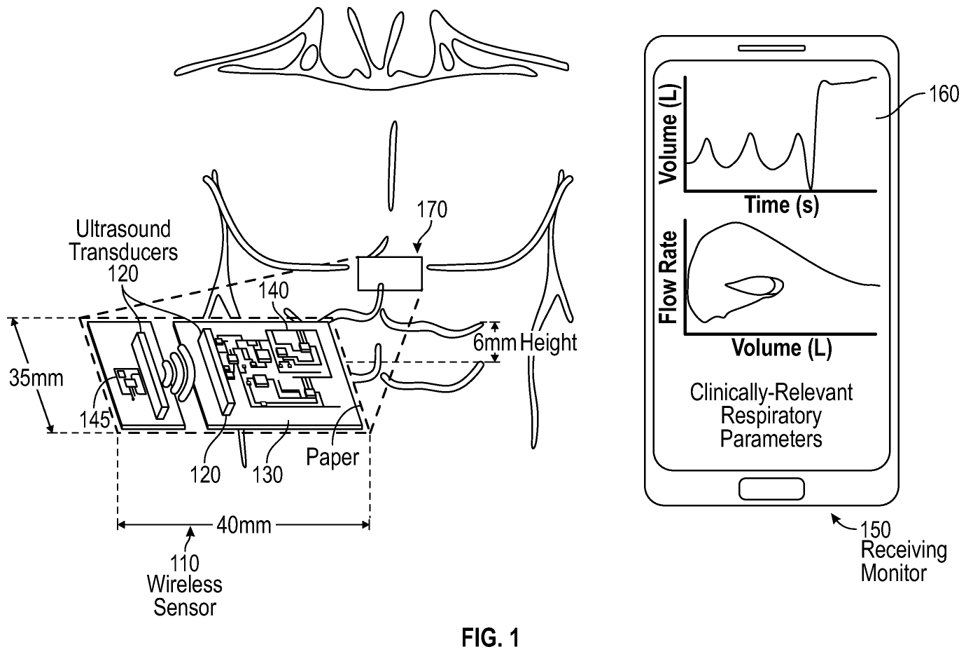

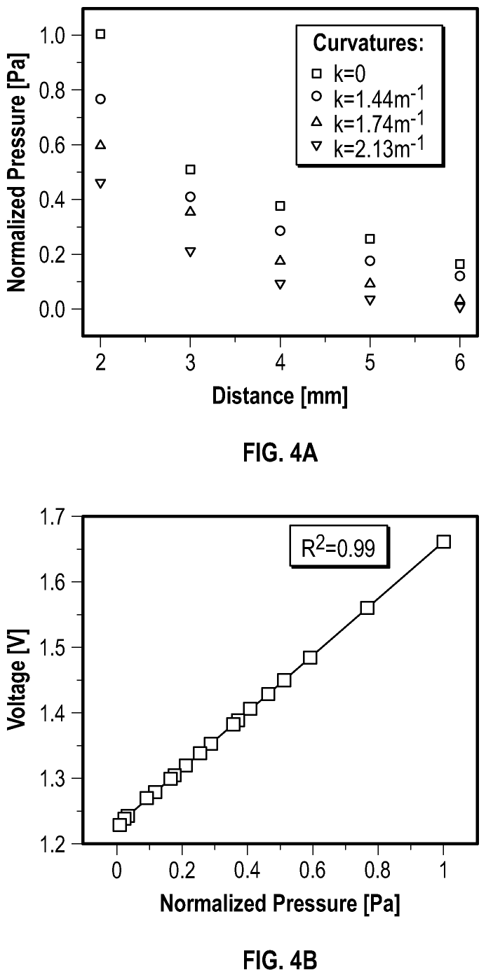

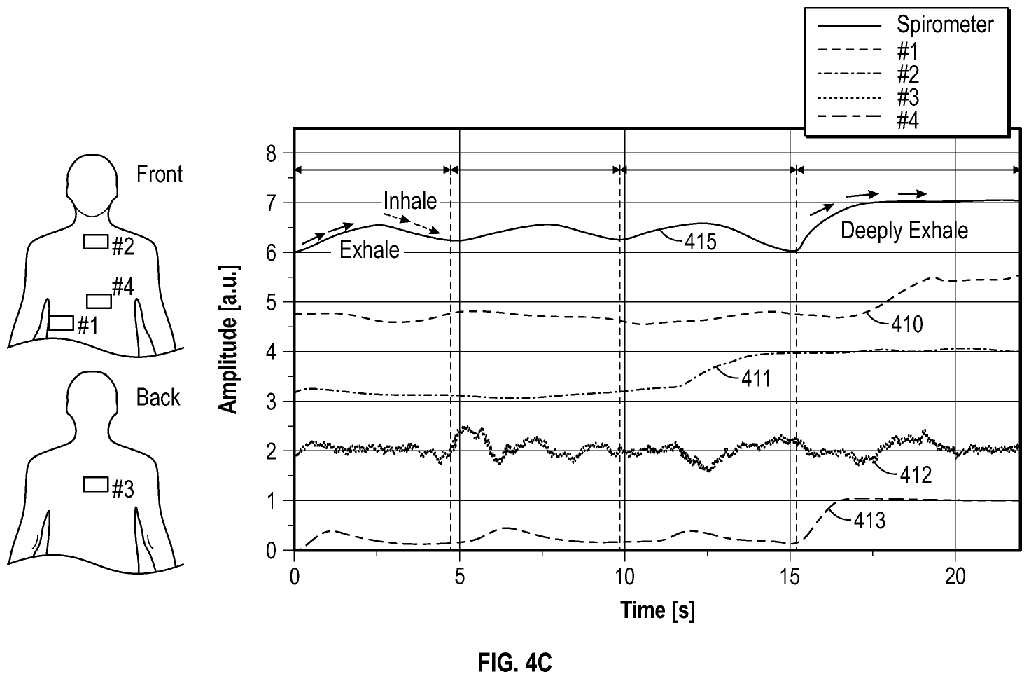

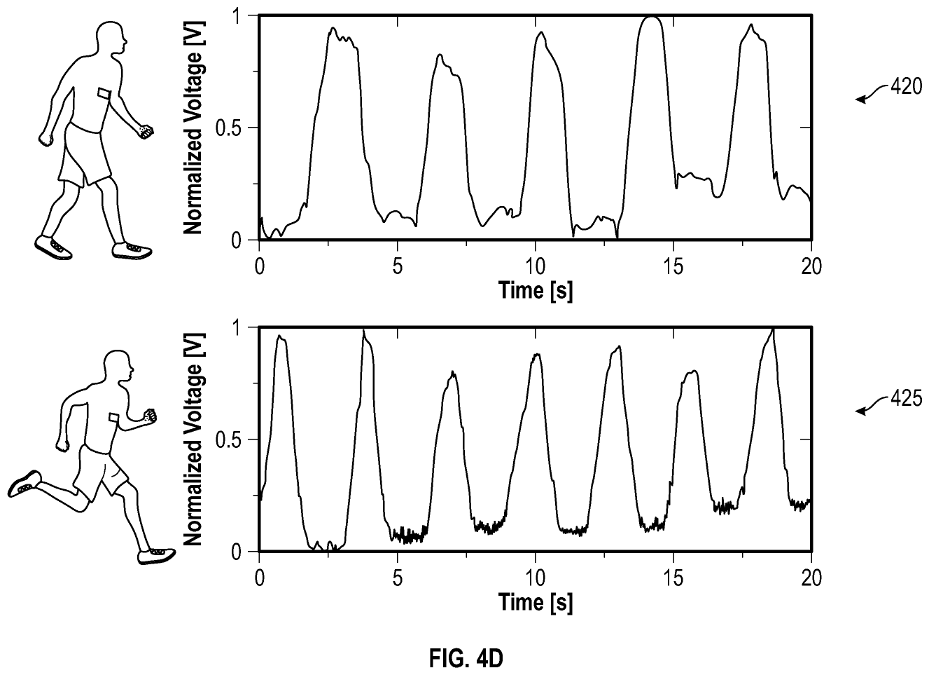

FIG. 1 shows a chest mounted wireless sensor 100 and receiving monitor 150 as may be employed in embodiments. As can be seen, the mounting location 170 of the sensor 110 may be on the chest of a wearer near the xyphoid process. Other mounting locations may also be employed. Some additional locations are described below and shown in FIG. 4C. The receiving monitor 150 may include a display 160 that provides graphical feedback in real-time as well as aggregated data for a single wearer or a cohort of wearers. As noted in FIG. 1, various clinically relevant respiratory parameters may be displayed. These parameters can include volume, flow rate, and breaths per minute. As shown in FIG. 1, ultrasound transducers 120 may be spaced apart from each other and may be mounted such that they can be used to determine pressure changes attributable to the expansion and contraction of the chest of a wearer. The wireless sensor 110 may include a paper substrate 130, control circuitry 140, and transducer circuitry 145. The receiving monitor 150 may also include internal circuitry to sore and perform processes as described herein.

The wireless sensor 110 may include control circuitry 140 designed to characterize respiration by determining the circumference changes in a person's chest occurring during respiration as measured by the change in distance between two transducers 120. The localized change of the circumference of the chest wall may serve to effectively emulate the lung volume behavior during respiration. When the diaphragm contracts, the intercostal muscles pull the ribs upwards causing the rib cage to be enlarged in the pump handle movement. In elevation, the anteroposterior diameter of the thorax increases and causes the lowermost ribs to swing outwards, which is called the bucket handle movement. Therefore, the horizontal enlargement of the thoracic cavity from the lifting of the front and sides of the ribs causes the circumference of the chest wall to increase during inspiration. During expiration, the diaphragm and intercostal muscles relax. The chest and abdomen passively return to a position determined by their anatomical elasticity, which results in a decrease in chest circumference.

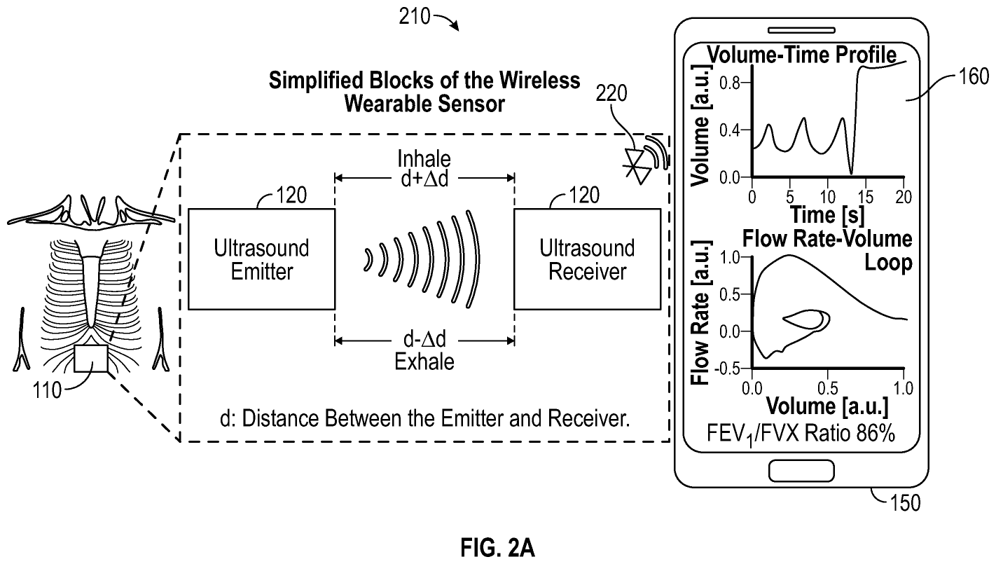

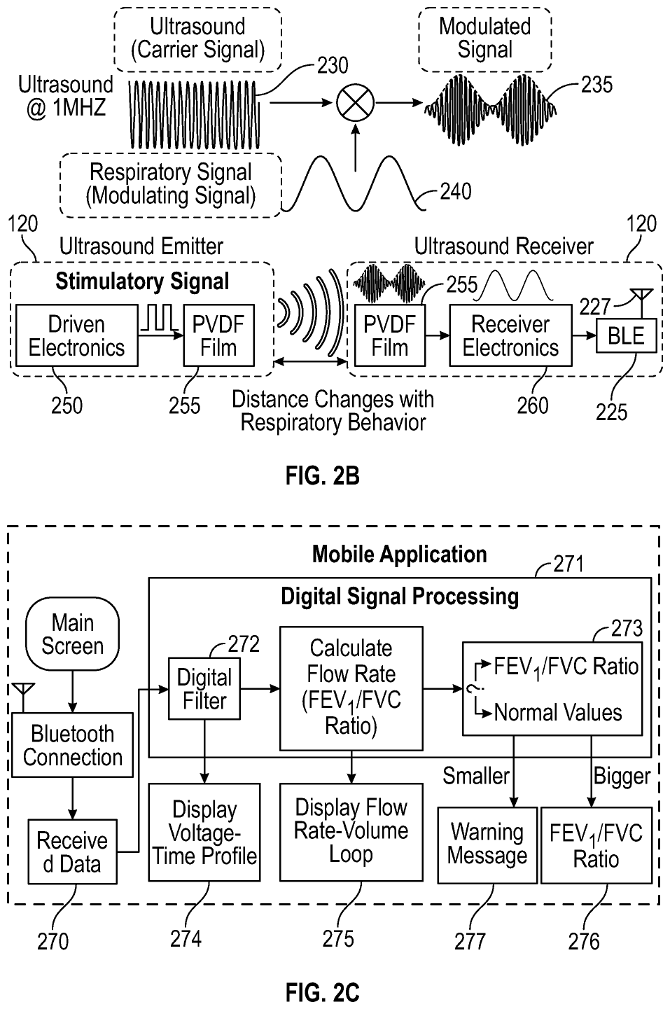

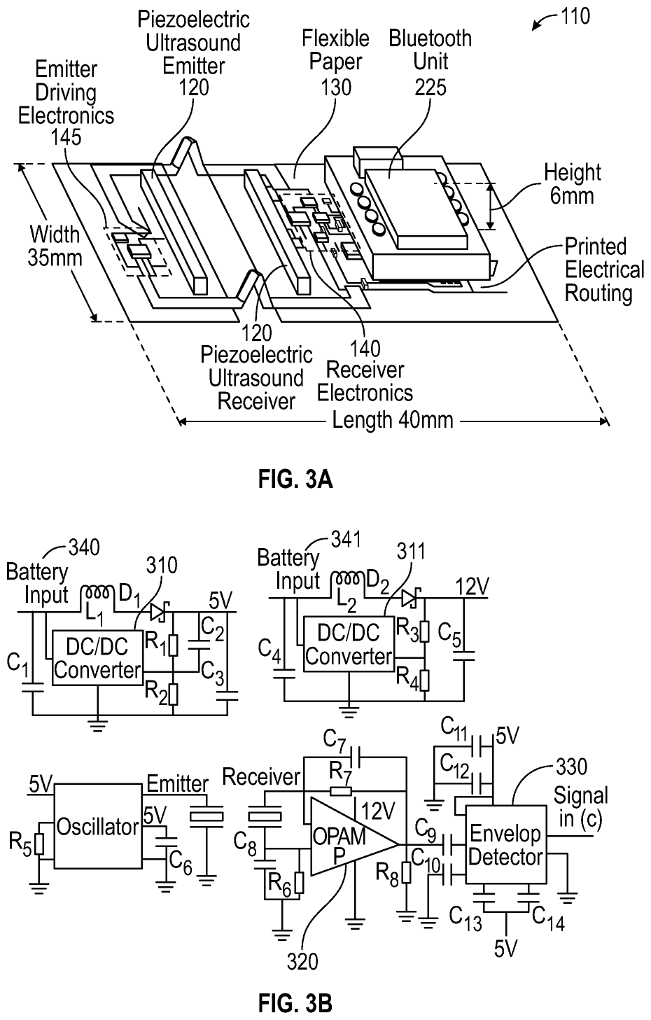

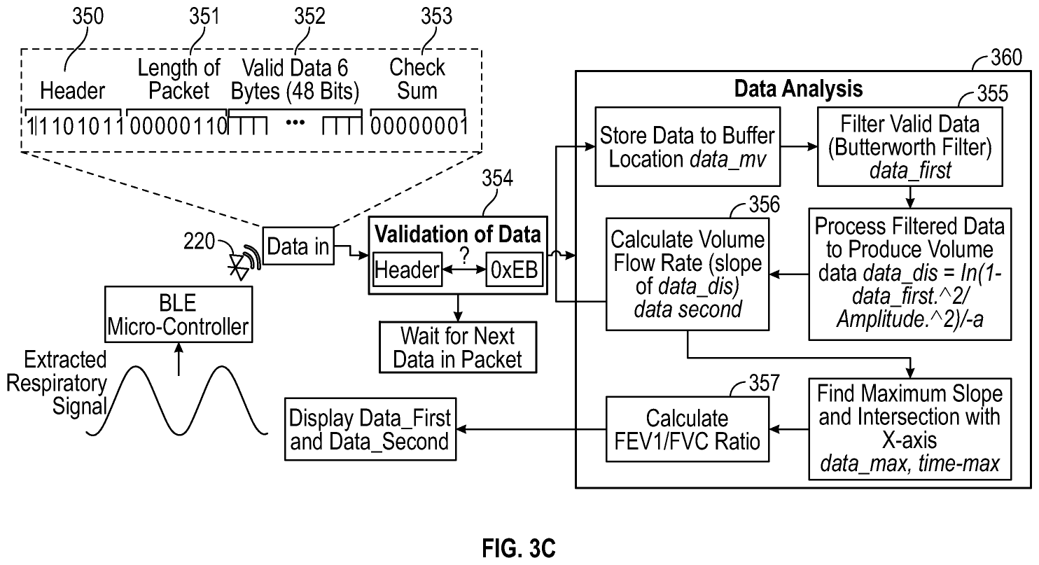

The wireless sensor 110 is preferably designed to measure the localized strain of chest wall circumference using the ultrasound transducers 120 or other sensing systems. The ultrasound transducers 120 may comprise an ultrasound emitter used to emit ultrasound and an ultrasound receiver to receive the distance-elapsed attenuated ultrasound. This ultrasound configuration is shown in an enlarged view in FIG. 3A. During inspiration, the ultrasound emitter and receiver move further apart, resulting in a more attenuated ultrasound signal whereas the emitter and receiver move closer together in expiration, resulting in an increased ultrasound signal. To generate the ultrasound, a non-polarized pulse stimulatory signal may be applied on a piezo-material, specifically Polyvinylidene Difluoride (PVDF) film. The ultrasound may be modulated by the respiratory signal and received by another PVDF receiver. After demodulation and amplification, the respiratory signal may be extracted by the control circuitry 140, and the digitized respiratory signal may then be wirelessly transmitted to the receiving monitor 150 by an onboard Bluetooth antenna in the control circuitry 140. An exemplary process flow is shown in FIG. 2B.

FIG. 2A shows a wearable wireless sensor 110 and distance process schematic 210, along with a receiving monitor 150, as may be employed in embodiments. The wearable wireless sensor 110, attached on the midline of the chest, measures a local strain of chest circumference as a function of time, to characterize respiratory behavior. In the distance process 210, the local stain is measured by modulated signal on an ultrasound carrier using ultrasound transducers 120. In the distance process 210, the temporal data is processed on board and transmitted to a receiving monitor 150 where an on-board application displays respiratory behavior plots 160 and computes clinically-relevant quantitative parameters. Ultrasound transducers 120 include an emitter and a receiver. In the distance process 210, during inhale and exhale a change in distance is measured and this distance change is reported via a wireless transmission 220 to the receiving monitor 150.

FIG. 2B shows additional details of the distance process schematic 210 as may be employed in embodiments—various operating processes of a sensor of embodiments are shown in FIG. 2B. In FIG. 2B, an ultrasound carrier 230, generated by an on-chip ultrasound emitter, is mixed with the respiratory signal 240, from the local strain of the chest circumference. The mixed modulated signal 235 is processed on board and transmitted to a smartphone via Bluetooth 220. Ultrasound transducers 120 are shown in FIG. 2B as either an emitter or a receiver. The emitter is shown to include driven electronics 250, PVDF film 255 and the receiver is shown with PVDF film 255, receiver electronics 260, Bluetooth electronics (wireless communication electronics) 225, and antenna 227. The distance between the two ultrasound transducers 120 changes with respiratory behavior over time.

FIG. 2C shows a schematic of process flow for a receiving monitor as may be employed in embodiments. The process flow of a receiving monitor may be managed by a locally run application as well as by a remote application in which the receiving monitor is a client. As can be seen in FIG. 2C, embodiments may include having the receiving monitor receive data 270 via a wireless connection and use DSP (Digital Signal Processing 271) filters 272 to calculate clinically-relevant respiratory behavior parameters: FEV1 (Forced Expiratory Volume delivered in the 1st second) and FVC (Forced Vital Capacity), and the FEV1/FVC ratio 273. The receiving monitor may also display temporal tracing and differential plots that show the respiratory behavior of a wearer in pseudo-real time, as may be employed in embodiments. These temporal tracing and differential plots can include voltage-time profile 274, a rate-volume loop 275, and a FEV₁/FVC ratio 276. Warning messages 277, may also be displayed in embodiments.

FIG. 3A shows a perspective view of components of a wearable wireless sensor 110 as may be employed in embodiments. As can be seen, in the wearable wireless sensor has a low profile, e.g., a height of 6 mm or less, and has a footprint of 40 mm×35 mm. The wearable wireless sensor 110 is also shown mounted on a paper substrate 130 an includes emitter driving electronics 145 to excite a piezoelectric Polyvinylidene Difluoride (PVDF) film to emit ultrasound waves via an ultrasound transducer 120, which is shown as a piezoelectric ultrasound emitter. Receiver electronics 140 are configured to convert received modulated ultrasound waves to electrical signals, and Bluetooth electronics 225 are configured to digitize the electrical signal and send them wirelessly. A second ultrasound transducer 120 is labelled as a piezoelectric ultrasound receiver in FIG. 3A, this transducer is configured to receive ultrasound signals from the opposing transducer in FIG. 3A.

In embodiments, a wearable sensor of approximately 40×35×6 mm3 may weigh approximately 6.5 g, which may include 2.7 g for batteries. Conductive electrical traces for sensors may be printed by a laser printer on a paper substrate such as OL177WS. During assembly, a pea-sized amount of silver epoxy (Atom A¬DUCT-1) may be squeezed out onto the printed circuit and may be spread evenly over the entire circuit. Also during assembly, hot air (˜200° C.) may be blown over the entire circuit in a lateral motion for 1 minute to dry out the silver epoxy and to melt the toner particles. Also, during assembly, the toner contains carbon and iron oxide, polypropylene, fumed silica and various minerals for triboelectrification, may be primarily composed of granulated plastic that is able to form a bond between the silver epoxy and the glossy paper in the shape of the circuit as it melts.

In embodiments, the paper substrate may be made with a layer of white clay called kaolin that fills between the paper fibers to produce a smooth surface. The organic mixture of silver epoxy may be unable to adhere to the kaolin surface, allowing easy removal of excess epoxy using a cotton ball. These two steps, during manufacture, may be repeated until the silver epoxy on the circuit lines became fully conductive. In embodiments, the finished lines may have a conductivity of 0.9Ω/cm with a width of 0.3 mm, and a different silver epoxy, having higher adhesion than the one above (Electron Microscopy Sciences, 12642-14 two-part conductive silver epoxy), may be used to mount the electrical components on the printed circuit. Still further, PVDF film may be attached to a rigid paper sheet using double-sided tape for standing support, and copper tape may be used to connect the PVDF films to the circuit due to its characteristic robust connection. Finally, copper tape may serve as the electrical bridges between the two pieces of the emitter and receiver pieces as well.

FIG. 3B shows circuit topology of a wireless sensor as may be employed in embodiments. As can be seen, the circuit topology can include two DC-to-DC converters 310, 311, which are configured and connected to provide power to corresponding electronic components. An op-amp 320 is shown and may be configured to amplify a received modulated signal, while an envelope detector 330 is also shown and may be configured in embodiments to extract respiratory behavior signals for subsequent outgoing wireless transmissions. Also shown in FIG. 3B are the battery inputs 340, 341, numerous capacitors, two inductors, several resistors, several diodes, and several capacitors.

In embodiments, a micropower DC/DC converter LM2704, from Texas Instruments Inc., may be employed to step up 3.7 V from a lithium battery to 5V or 12 V, with current limits of 120 mA or 40 mA, respectively. A voltage-controlled oscillator LTC6990, from Analog Devices Inc. may be employed to excite the PVDF film of the emitter in order to generate a 1 MHz ultrasound signal. Another PVDF film may be employed to detect the ultrasound signal, which has been modulated by a respiratory signal. A low noise, FET-input operational amplifier (OPA657 from Texas Instruments Inc.) may also be employed to filter and process the received modulated signal. A single OPA657 stage, offering 1 600 V/V(64 dB) for a 1 MHz signal, may also be employed. This op/amp or other selected op/amp may be characterized by a high gain-bandwidth product (1.6 GHz) and low voltage noise JFET-input stage. In embodiments, a very low-level signal can be significantly amplified by using amplification that comprises an envelope detector (ADL 55 11, ADI) to extract a respiratory signal. This signal may then be sent to a wireless transmitter, e.g., Bluetooth Low Energy Nano V2 (RedBearLab) using a 100 Hz sampling frequency with ultra-low power consumption. The BLE device may use an onboard analog to digital converter (ADC) with 12-bit resolution to digitize the analog input signal before transmitting it to external receiving devices for analysis.

FIG. 3C shows operational process flows related to wireless sensor data gathering and subsequent data as may be employed in embodiments. As shown in FIG. 3C, in embodiments, the extracted respiratory behavior signal may be digitized and coded by onboard micro-controller into four kinds of strings: header 350, length of the packet 351, valid data 352, and checksum 353. These strings may be sent, via a wireless protocol 220, to an application specific integrated circuit (ASIC) or an application running on a receiving monitor. The ASIC/receiving monitor may analyze the transmitted data including checking the header for validity 354, filtering the valid data 355, and extracting the clinically-relevant parameters 356 and 357. Other processes may also be performed as part of the data analysis 360.

In embodiments, a receiving monitor may be configured to receive a wireless signal from the sensor and filters it by Butterworth low-pass filter with a cut-off of 0.5 Hz to display primarily two graphs: the temporal trace of the voltage corresponding to volume vs. time and the flow rate vs. volume plot. The second graph may be used to determine the FEV1/FVC ratio, which may then be compared to the nominal value of 0.75. The American Thoracic Society has identified the back-extrapolation method as the most consistent and accepted technique for determining the start point and has recommended its use for every calculation of FEV1. In certain embodiments, the extrapolated volume may not be higher than 5% of the FVC or 150 mL, whichever number is higher.

During testing or calibration of a wearable sensor, in embodiments, rather than using extrapolated volume via only spirometry, the extrapolated output voltage of a sensor may be defined relationally. This correlation may be made between the output of the wearable wireless sensor with a unit of voltage and the output of the spirometer test with a unit of milliliter or liter. To determine the extrapolated voltage, a differential analysis of the temporal output may be used to find the section of the deep expiration curve with the greatest tangential slope. Then, in some embodiments, the most significant value, the previous data point, and the following data point may be used to plot a trend line. From the equation of the trend line, the intersection with the x-axis can be found. This x-intercept may then be used as the new start time (t=0) and the corresponding y-axis value y₀ on the voltage-time graph may be used to extrapolate voltage. The point on x-axis which is one second afterward is designated t=1, with the corresponding y-axis value designated as y₁. Regarding the maximum voltage as the FVC and FEV1 as y₁-y₀ with a unit of voltage, then

(FEV₁/FVC) ratio=(y₁-yₒ/maximum voltage)*100%

When the FEV₁/FVC ratio reaches below 75%, a warning message may show on the mobile phone screen or other receiving monitor to inform the user of the abnormal respiratory behavior. FIG. 4B shows the output voltage of an exemplary wearable sensor as a function of normalized pressure at the receiver, demonstrating suitable linearity.

The data analysis in embodiments may be conducted using the following format and algorithm.

### Algorithm of Custom-made Mobile App

Packet Format:

Android Application Signal Processing Algorithm

| | | |
|---|---|---|
| 1: | Init: data ← data from sensor | //save bitstream data sent in 16 |

binary system.

| | |
|---|---|
| 2: | Init: package_len ← length of effective data; data_mv ← save valid data; data_time ← |

save sample time

size ← record the number of packets; length ← the number of packets processed

currently

| | |
|---|---|
| 3: | while length<size do |

| | |
|---|---|
| 4: | Check: Check the head of packet must be ‘0xeb’ |
| 5: | Through Butterworth filter; |

| | | | |
|---|---|---|---|
| 7: | Init: az bz ← read filter coefficients from ‘butter_coe.text’ | //Coefficients | file |

created in MATLAB

data_first ← save the filtered data

| | |
|---|---|
| 8: | Deal: data_first(t)=bz₀*data_mv(t) + bz₁*data_mv(t−1) + ... +bz_{order}*data_mv(t−order) |

| | |
|---|---|
| − az₁*data_first(t−1) − az₂*data_first(t−2) − ... −az_{order}*data_first(t−order) | //order |

← filter order

| | |
|---|---|
| 9: | Init: deal_dis ← save transition data; data_second ← savesecond processed data |

(represents slope)

| | |
|---|---|
| 10: | Second Deal: |

| | |
|---|---|
| | deal_dis(t) ←ln(\|1−(data_first(t).\hat{ }2)/(Amplitude_Level\hat{ }2)\|)/(−a); |
| 11: | data_second(t) ← (deal_dist(t−1) − deal_dis(t))/data_time(t)−data_time(t−1)) |

| | |
|---|---|
| 12: | Calculate FEV1 and FEV1/FVC: |

| | |
|---|---|
| 13: | Init: max_Data ← maximum of filtered data with t>0.5s; max_Time ← the time of |

max_Data;

min_Time ← the time of trough value before maximum data;
delta_Time ← x-intercept of maximum slope plus one; time_Plus ← truly

effective intersection

| | |
|---|---|
| 14: | Deal: for t in [data_second.size( )−pakage_len/2, data_second.size( )] do |

| | |
|---|---|
| 15: | if (max_data<data_second(t)) max_data ← data_second(+) |
| 16: | max_time ← t |

| | |
|---|---|
| 17: | end of for |
| 19: | for t<max_Time do |

| | |
|---|---|
| 20: | if(data_first(t)>data_first.get(t−1)) |

| | |
|---|---|
| 21: | t−−; |

| | |
|---|---|
| 22: | end of for |
| 23: | min_time ← t |
| 24: | for t in [min_Time, max_time] do |

| | |
|---|---|
| 25: | Find the maximum slope point. |

| | |
|---|---|
| 26: | end of for |
| 27: | delta_time ← 1+ x-intercept of maximum slope |
| | Time_Plus ← closest valid point to delta_Time |

| | |
|---|---|
| 28: | FEV1 ← time_Plus |
| 29: | FEV1/FVC ← FEV1/max_Data |

| | |
|---|---|
| 30: | end of while |
| 31: | Show: display waveform. |

FIG. 4A shows normalized ultrasound pressure as a function of the distance between the ultrasound emitter and receiver as may be experienced in embodiments. On the surfaces of different curvatures, the pressure decreases as the distance increases because the attenuation of ultrasound wave increases with distance. The table of FIG. 4A shows that this trend exhibits a consistent pattern on different curvatures, e.g., flat (k=0) and largely curved (k of up to 2.13 m−1). This relationship between pressure, curvature of the wearer location, and distance between the ultrasound transducers may be employed when calibrating a sensor and receiving monitor system. The emitter and the receiver may be symmetrically located on either side of the midway of a human chest, thus the common-mode movement cancels out each other. Assuming little misalignment exists between PVDF films of the emitter and the receiver, the collected signal maintains stable throughout the body movement, highlighting the wear-ability of the wearable wireless sensor.

The functionality of the wearable wireless sensor should maintain at a given curvature on the body where the sensor is located. As the distance changes between the emitter and receiver, the corresponding ultrasound pressure changes at curvatures of 0 m⁻¹, 1.44 m⁻¹, 1.74 m⁻¹ and 2.13 m⁻¹, as shown in FIG. 4A. The curvatures experiments cover the range of the chest wall curvatures effectively.

FIG. 4B shows output voltage of a wireless sensor as a function of applied normalized pressure, as may be employed in embodiments. This normalized pressure is applied to the PVDF film of ultrasound transducers. As can be seen in FIG. 4B, in some embodiments, the output of sensor, voltage versus pressure, may show a very linear response to the normalized pressure. In other words, a linear relationship may exist in the output voltage function when considering the distance between an ultrasound emitter and an ultrasound receiver.

FIG. 4C shows amplitude versus time outputs of a wireless sensor mounted on four locations of the chest as may be employed in embodiments. These four outputs 410-413 are further compared to spirometer output 415. As can be seen, a front mid chest location, no. 4, provides amplitude versus time outputs most consistent with spirometer outputs. These outputs were determined using eight volunteers. In this calibration, each volunteer, in a sitting position with a nose-clip on, completed three normal breathing cycles through the mouthpiece. On the fourth cycle, the volunteer took a deep breath in and exhaled thoroughly in as brief a time as possible to maximize the airflow in the first second and keep exhaling at least 6 seconds to ensure a complete test result. During the procedure, volunteers were asked to keep their backs straight. Keeping backs straight allows the rib cage not to weigh on the abdomen and the abdominal wall to maximize the accuracy. After proper relaxation, the wearable wireless sensors were placed at the designated location, and the volunteers repeated the procedure. Due to its compact size and wear-ability, the wearable wireless sensor has limited effect on the spirometry test, and so both sensors were used simultaneously to eliminate as many variables as possible. The most prominent artifact from human body movement includes vertical up and down motion when the subject walks or run.

FIG. 4D shows normalized output voltage v. time for a wireless sensor when a wearer is walking 420 and running 425, as may be employed in embodiments. This dynamic characterization is shown for walking with a speed of 1.2 m/s for 20 seconds, and running with a speed of 2 m/s for 20 seconds. FIG. 4D shows light movement of body has little impact on the quality and the distinguishability of data. The motion artifact may impact large and heavy wearable sensors, due to the moment of inertia, but the lightweight 6.5 g sensor showed little motional artifact.

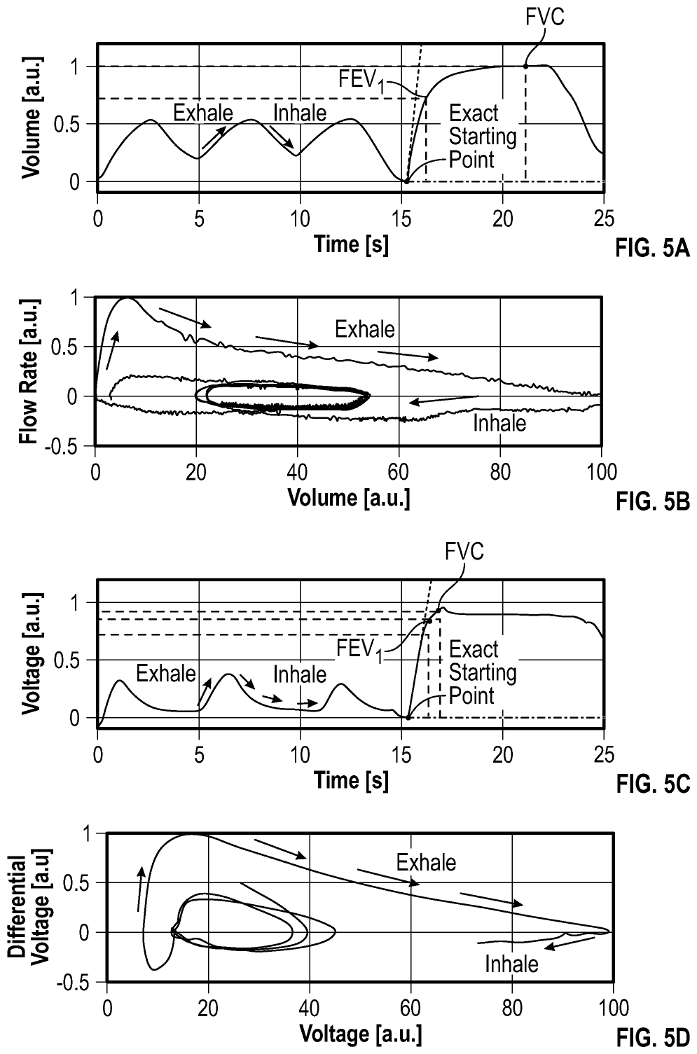

FIG. 5A shows a respiratory behavior plot of volume v. time using a spirometer for reference as may be employed in embodiments. FIG. 5B shows a respiratory behavior plot of flow rate v. volume using a spirometer for reference as may be employed in embodiments. FIG. 5C shows a respiratory behavior plot using a wearable wireless sensor of voltage v. time as may be employed in embodiments. FIG. 5D shows a respiratory behavior plot using a wearable wireless sensor of differential voltage v. voltage as may be employed in embodiments. In FIGS. 5A and 5C volume-time tracing with marked FEV1, FVC, and extrapolated volume are provided, whereas in FIG. 5B and 5D flow rate vs. volume for spirometer and wearable wireless sensor are provided.

In FIGS. 5A-5D, clinically-relevant parameters, including FEV1 and FVC, are computed from spirometer and sensor, respectively, and may be used for calibration in embodiments. As can be seen in FIGS. 5A-5D the temporal output profile of the sensor, FIG. 5C, has a strong correlation with that of the spirometer, FIG. 5A. Both start with a general expiration pattern, shown by the tracings both trending upwards and exhibiting the similar respiration behaviors. The sensor illustrates inhalation behavior slightly earlier than spirometer does, which is considered to be attributable to the inherent discrepancy between direct and indirect measurements of lung volume. The spirometer directly measures the flow out of the lungs through the mouthpiece whereas the sensor indirectly measures the lung volume via the circumference change of the chest wall. Several reported the circumference change of the chest wall occurs before the start of inspiration and expiration due to the mechanics of respiration. For the fourth deep expiration, since the exhale was purposely completed as fast as possible, very little time delay was observed in both temporal plots. The temporal output of the sensor was used to obtain the volume flow rate graph shown in FIG. 5D, which is comparable to the flow-volume tracing of the spirometer in FIG. 5B. Both figures start with the general respiration (small loops in the figures) and once a forced expiration is performed, the curve rapidly mounts to a peak, known as the peak expiratory flow (PEF). After the PEF, the curve descends, representing the decreasing flow as more air is exhaled. A straight or a convex tracing from PEF to FVC point (the greatest intercept on the x-axis) would indicate non-pathological respiratory behavior. In results from both the spirometer in FIG. 5B and the sensor in FIG. 5D, a convex tracing was observed, meaning the respiratory behavior for this specific example is normal. In results from both the spirometer in FIG. 5B and the wearable wireless sensor in FIG. 5D a concave tracing was observed representing the respiratory behavior of the specific test on the volunteer is sub-optimal. For the results displayed in the figures, the FEV1/FVC ratios of 71% and 72.5% were achieved by spirometer and the sensor during the attempt, respectively, which confirms the sub-optimal respiratory performance of this volunteer was detected by both spirometer and the sensor. When the FEV1/FVC ratio is smaller than the pre-set value in the custom-made app (75%), an alerting message will display on the screen of the mobile phone. The FEV1/FVC ratios of 86% and 87.94% may be targeted by spirometer and the sensor, respectively, supporting the sensor is capable of measuring the normal respiratory behaviors.

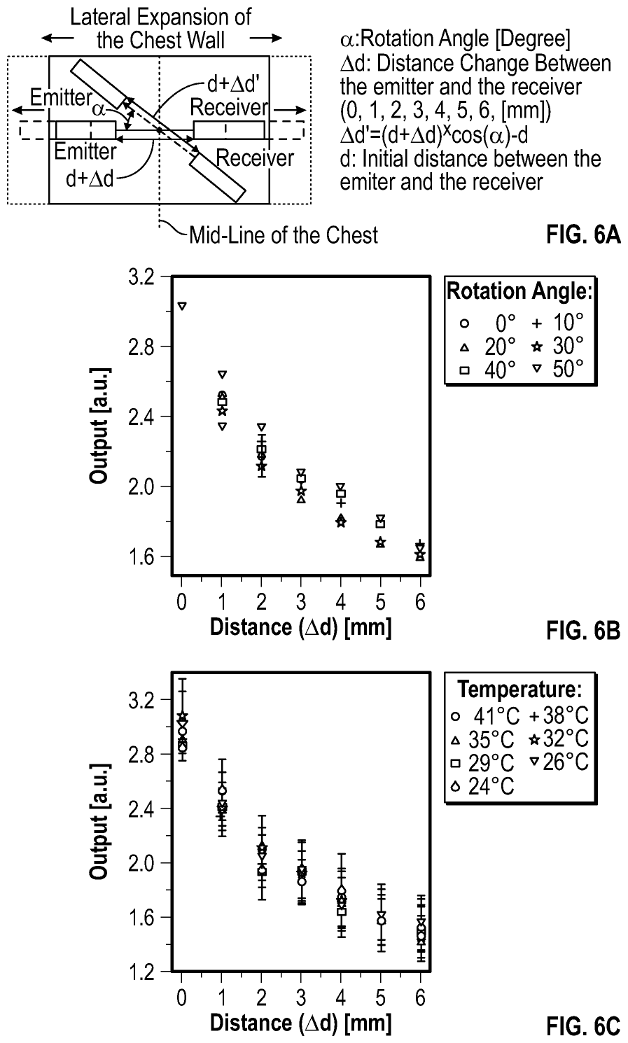

FIG. 6A shows calibration testing of a wearable wireless sensor at different rotation angles as may be employed in embodiments. FIG. 6A shows a schematic of locations of a wearable wireless sensor being calibrated at different rotation angles, with the increase of the distance between the emitter and the receiver from 0 mm to 6 mm (at 1 mm distance interval in the horizontal direction) to demonstrate continued functionality with sub-optimal sensor placement is shown. As shown in FIG. 6B, at a given rotation angle, the output decreases as the distance between the emitter and the receiver increases. The sensitivity, the slope, decreases as the angle increases because the effective distance between the emitter and the receiver decreases. As shown in FIG. 6C, ambient temperature may have smaller effects than other variables when calibrating a wearable sensor and receiving monitor system of embodiments. Here, the sensor was tested under different temperatures, ranging from 24° C. to 41° C., and demonstrated a relative insensitivity, within this range, to ambient temperature change.

More specifically, in embodiments, the output signal can obtain suitable results from a horizontally located wearable wireless sensor because the lateral expansion of the chest wall is more significant at this sensor orientation. A user, however, may not place the wearable wireless sensor exactly where it is designed to be, resulting in finite error at the sensor output. Accordingly, the wearable wireless sensor was tested at different angles with the increase of the distance between the emitter and the receiver ranging from 0 mm to 6 mm with a 1 mm interval in the horizontal direction to verify the sensor is capable of working properly in non-ideal positions. When the rotation angle is zero, the horizontal movement equals the distance change between the emitter and the receiver, as shown in FIG. 6A. The effective distance between the emitter and the receiver is a function of the rotation angle. As the rotation angle increases, the effective distance decreases, thus the sensitivity, the slope of the output vs distance, decreases, as shown in FIG. 6B. All the starting points have been overlapped together for easy comparison of the effect of the rotation angles on the wearable wireless sensor. The experimental results show the sensitivity suffers by 5%, 8%, 3%, 2%, and 1% from human-error induced rotation of 10°, 20°, 30°, 40°, and 50°, respectively. The loss of sensitivity impacts the critically-relevant parameter, FEV1/FVC, by 8%, supporting that the wearable wireless sensor offers acceptable performance within a reasonable angle of rotation.

The body itself provides a rather isothermal setting, yet the operating temperature of wearable wireless sensor may change as a function of external temperature. The sensor was tested under temperatures ranging from 24° C. to 41° C. to study the temperature sensitivity. The outputs of the sensor were recorded at the distance of 0-6 mm from 24° C. to 41° C., as shown in FIG. 6C. While slightly varying outputs were observed, the outputs variation was within measurement errors, demonstrating that the temperature effect on our sensor is very limited.

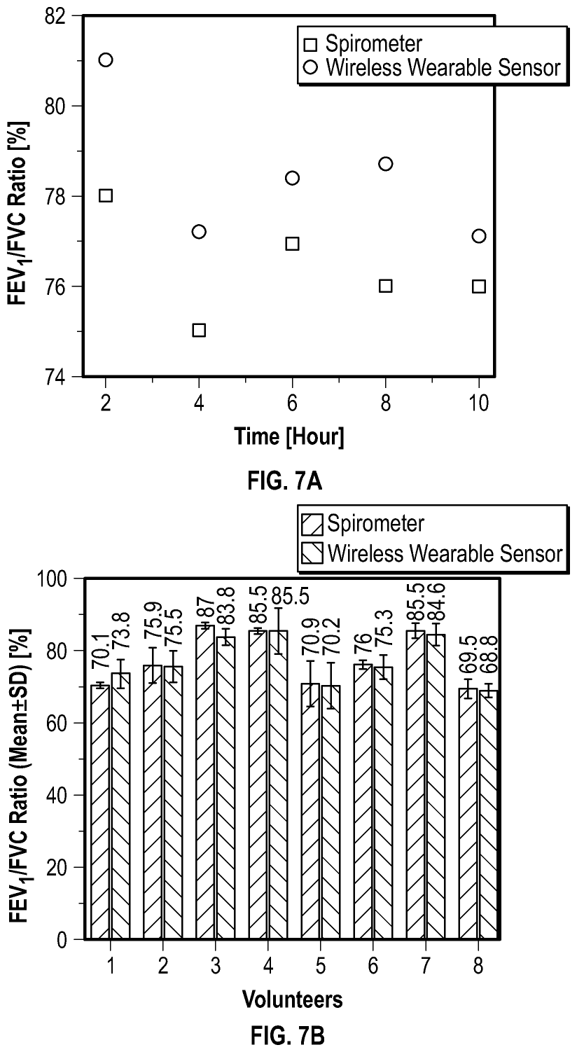

FIG. 7A shows the FEV1/FVC ratio output of a wearable wireless sensor at 2-hours, 4-hours, 6-hours, 8-hours, and 10-hours, as may be employed in embodiments. FIG. 7B shows testing results of the FEV1/FVC ratio of a spirometer and a wearable wireless sensor of embodiments for eight different wearers. In FIG. 7B, the mean differences between the spirometer and the sensor range from 0.00% to 4.25%, demonstrating suitable correlation between the spirometer and the sensor calibration, as may be employed in embodiments.

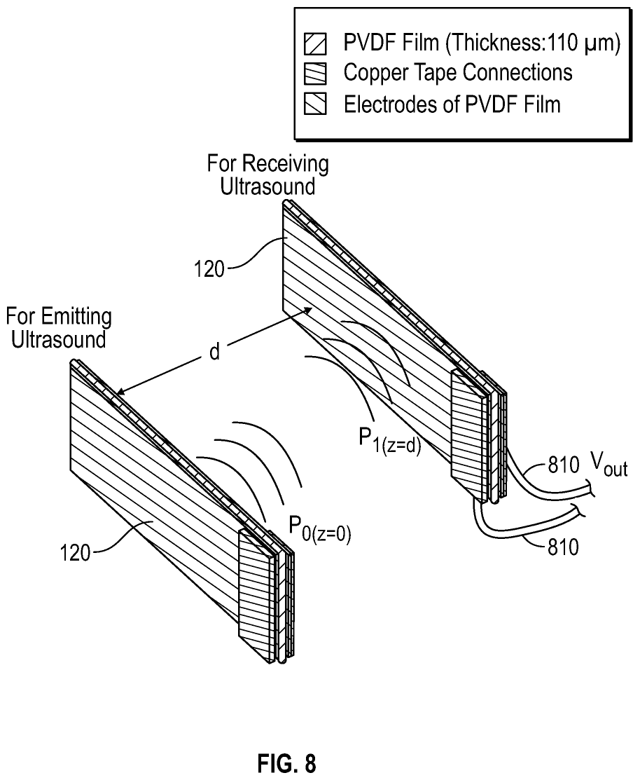

FIG. 8 shows two PVDF films that may be mounted on a wearable wireless sensor in embodiments. As can be seen, each ultrasound transducer 120 comprises a PVDF film and copper tape connections. The receiving transducer also comprises traces 810 for output voltage. As the distance between the transducers grow signal attenuation will result as ultrasound pressure attenuation increases with travelling distance. One is for emitting ultrasound and the ultrasound pressure is at zero travelling distance. The ultrasound pressure reaches another PVDF film used to receive attenuated ultrasound, at a distance of d. The corresponding voltage output is corelated to as a function of d that changes as a function of respiration. The overall ultrasound attenuation is characterized by the following exponential decrease of the pressure amplitude P with the travelling distance z:

P₁=Pₒ*e^{-alpha*z}

where P₁ is the pressure at z, Pₒ is the pressure at z=0, alpha (expressed in cm−1) is the pressure frequency-dependent attenuation coefficient.

The output voltage generated by the PVDF film is given by:

Vₒᵤₜ=g33×P₁xt

where g33 is ((V/m)/(N/m²)) and is the piezo stress constant for electric fields induced in three directions by a stress of one Pascall applied along the “3” axis. The typical value of PVDF film is −330×10⁻³ ((V/m)/(N/m²)) and t is the thickness of the PVDF film, which is approximately 110 μm.

Various features, steps, processes, components, and subcomponents, as may be employed in embodiments, are provided herein. These features, steps, processes, components, subcomponents, partial steps, systems, devices, etc. may be adjusted, combined and modified in various fashions and various ways among and between the teachings and figures provided herein, as well as in other ways not specifically described herein but consistent with the teachings and discussion of this disclosure.

The terminology used herein is for the purpose of describing particular embodiments only and is not intended to be limiting. As used herein, the singular forms “a,” “an” and “the” are intended to include plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The use of any and all examples, or exemplary language (e.g., “such as”) provided herein, is intended merely to better illuminate and does not pose a limitation on scope unless otherwise claimed. No language in the specification should be construed as indicating any non-claimed element as essential.

As used herein, the terms “about” or “approximately” in reference to a recited numeric value, including for example, whole numbers, fractions, and/or percentages, generally indicates that the recited numeric value encompasses a range of numerical values (e.g., +/−5% to 10% of the recited value) that one of ordinary skill in the art would consider equivalent to the recited value (e.g., performing substantially the same function, acting in substantially the same way, and/or having substantially the same result). As used herein, the terms “about” or “approximately” in reference to a recited non-numeric parameter generally indicates that the recited non-numeric parameter encompasses a range of parameters that one of ordinary skill in the art would consider equivalent to the recited parameter (e.g., performing substantially the same function, acting in substantially the same way, and/or having substantially the same result).

Recitation of ranges of values herein are merely intended to serve as a shorthand method of referring individually to each separate value falling within the range, unless otherwise indicated herein, and each separate value is incorporated into the specification as if it were individually recited herein.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

Certain embodiments may be implemented as a computer process, a computing system or as an article of manufacture such as a computer program product of computer readable media. The computer program product may be a computer storage medium readable by a computer system and encoding computer program instructions for executing a computer process.

The corresponding structures, material, acts, and equivalents of any means or steps plus function elements in the claims are intended to include any structure, material or act for performing the function in combination with other claimed elements. The description of certain embodiments has been presented for purposes of illustration and description, but is not intended to be exhaustive or limited to the form disclosed. Many modifications and variations will be apparent to those of ordinary skill without departing from the scope and spirit of the disclosure. These embodiments were chosen and described in order to best explain the principles of the disclosure and practical application, and to enable others of ordinary skill in the art to understand possible embodiments with various modifications as are suited to the particular use contemplated.

What is claimed is:

1. A system for monitoring respiration behavior of a wearer, comprising:
a wireless sensor, the sensor configured to output respiratory behavior data of a wearer; and
a receiving monitor, the receiving monitor configured to wirelessly receive the respiratory behavior data from the wireless sensor and to determine respiratory behavior of the wearer,
wherein the wireless sensor is further configured to be surface mounted on a torso of a wearer, the wireless sensor is further configured to generate an ultrasound signal, and the wireless sensor does not completely encircle the torso of the wearer.

2. The system of claim 1 wherein the receiving monitor is configured to convert linear strain data embedded in the output respiratory behavior data to lung volume change by determining the change in ultrasound pressure as a function of the distance between a first ultrasound transducer and a second ultrasound transducer of the wireless sensor.

3. The system of claim 1 wherein the wireless sensor is further configured to generate an ultrasound signal by applying a non-polarized pulse stimulatory signal on a piezo-material.

4. The system of claim 3 wherein the piezo-material is Polyvinylidene Difluoride (PVDF) film and the ultrasound signal is modulated by a respiratory signal and received by a PVDF receiver of the wireless sensor.

5. The system of claim 1 wherein the wireless sensor comprises a first ultrasound transducer and a second ultrasound transducer, the first transducer configured as an emitter and the second transducer configured as a receiver, the first transducer further configured to modulate a respiratory signal onto an ultrasound signal and create a modulated signal.

6. The system of claim 5 wherein the first transducer is further configured to send the modulated signal to the second transducer.

7. The system of claim 1 wherein the receiving monitor is configured to determine respiratory flow rate of the wearer using the received respiratory behavior data.

8. A system for monitoring respiration behavior of a wearer, comprising:
a wearable sensor, the sensor configured to output respiratory behavior data of a wearer; and
a receiving monitor, the receiving monitor configured to receive the respiratory behavior data from the wearable sensor and to determine respiratory behavior of the wearer,
wherein the wearable sensor is further configured to be surface mounted on a torso of a wearer, is further configured to generate an ultrasound signal, and the wearable sensor does not completely encircle the torso of the wearer.

9. The system of claim 8 wherein the receiving monitor is configured to convert linear strain data embedded in the output respiratory behavior data to lung volume change by determining the change in ultrasound pressure as a function of the distance between a first ultrasound transducer and a second ultrasound transducer of the wearable sensor.

10. The system of claim 8 wherein the wearable sensor is further configured to generate an ultrasound signal by applying a non-polarized pulse stimulatory signal on a piezo-material.

11. The system of claim 10 wherein the piezo-material is Polyvinylidene Difluoride (PVDF) film and the ultrasound signal is modulated by a respiratory signal and received by a PVDF receiver of the wearable sensor.

12. The system of claim 8 wherein the wearable sensor comprises a first ultrasound transducer and a second ultrasound transducer, the first transducer configured as an emitter and the second transducer configured as a receiver, the first transducer further configured to modulate a respiratory signal onto an ultrasound signal and create a modulated signal.

13. The system of claim 12 wherein the first transducer is further configured to send the modulated signal to the second transducer.

14. The system of claim 8 wherein the receiving monitor is configured to determine respiratory flow rate of the wearer using the received respiratory behavior data.

15. A system for monitoring respiration behavior of a wearer, comprising:
a wireless sensor, the wireless sensor configured to output respiratory behavior data of a wearer; and
a receiving monitor, the receiving monitor configured to wirelessly receive the respiratory behavior data from the wireless sensor and to determine respiratory behavior of the wearer,
wherein the wireless sensor is further configured to be surface mounted on a torso of a wearer and the wireless sensor comprises a plurality of ultrasound transducers, the transducers spaced a variable distance apart from each other, the distance changing during lung volume change of the wearer.

16. The system of claim 15 wherein the receiving monitor is configured to convert linear strain data embedded in the output respiratory behavior data to lung volume change by determining the change in ultrasound pressure as a function of the distance between the transducers.

17. The system of claim 15 wherein the wireless sensor is further configured to generate an ultrasound signal by applying a non-polarized pulse stimulatory signal on a piezo-material.

18. The system of claim 17 wherein the piezo-material is Polyvinylidene Difluoride (PVDF) film and the ultrasound signal is modulated by a respiratory signal and received by a PVDF receiver of the wireless sensor.

19. The system of claim 15 wherein the plurality of transducers comprises a first ultrasound transducer and a second ultrasound transducer, the first transducer configured as an emitter and the second transducer configured as a receiver, the first transducer further configured to modulate a respiratory signal onto an ultrasound signal and create a modulated signal.

20. The system of claim 19 wherein the first transducer is further configured to send the modulated signal to the second transducer.
